(19) United States
(12) Patent Application Publication
(10) Pub. No.: US 2012/0071911 A1
(43) Pub. Date: Mar. 22, 2012

(54) SPHERICAL HELIX EMBOLIC COILS FOR THE TREATMENT OF CEREBRAL ANEURYSMS

(75) Inventors: Chandramouli Sadasivan, Miami Beach, FL (US); Baruch B Lieber, South Setauket, NY (US)

(73) Assignee: UNIVERSITY OF MIAMI, Miami, FL (US)

(21) Appl. No.: 13/320,566

(22) PCT Filed: May 20, 2009
(86) PCT No.: PCT/US2009/044652

§ 371 Date: Nov. 15, 2011

(51) Int. Cl. A61M 29/00

(52) U.S. Cl. 606/191

(57) ABSTRACT

A method and device for occluding an aneurysm. The device may include a wire having a shape memory. The wire may define a coil. The wire may further define a first substantially spherical helix and a second substantially spherical helix nested within the first substantially spherical helix. A catheter may be included, being releaseably engageable to the wire, the wire being at least partially disposed within the catheter.

### FIELD OF THE INVENTION

The present invention relates to a method and device for occluding aneurysms in blood vessels.

### BACKGROUND OF THE INVENTION

Aneurysms are blood-filled dilations of a blood vessel generally caused by disease or weakening of the blood vessel wall. Blood may flow into an opening defined by the blood vessel wall, called the neck, causing the aneurysm to expand. Aneurysms commonly occur at bifurcation points of the major arteries of the brain. The wall of a brain aneurysm may progressively thin, leading to an increased risk of rupture causing hemorrhagic stroke or even sudden death. There are about 30,000 to 40,000 cases of aneurysmal rupture per year in the United States, accounting for about 5% of all strokes. The prognosis after aneurysmal rupture is poor; the 30-day mortality rate is approximately 45% and a positive functional outcome is achieved in only 40-50% of survivors.

One emerging method to combat aneurysmal rupture involves endovascular occlusion of the aneurysm. Present endovascular aneurysm treatments include packing the aneurysm with metallic coils and partially occluding the aneurysm. While inside an endovascular catheter, the coil may define a two-dimensional linear wire. The coil may be further engaged to an endovascular delivery mechanism by methods known in the art, such that after the coil has been deployed into the aneurysm, it can be safely disengaged from a delivery mechanism. Once deployed into the aneurysm, the coil may define a variety of configurations. These configurations have evolved from two-dimensional structures to three-dimensional structures, which are typically created by randomly winding the coil within the aneurysm.

One drawback of treating aneurysms with embolic coils is that the coil mass within the aneurysm tends to compact under the repeated pulsatile impingement of blood. This may be especially prevalent when the coil mass has been randomly packed in the aneurysm, which leaves a large volume of interstitial space into which blood can flow and potentially lead to re-canalization or re-growth of the aneurysm over a period of time. As a result, patients treated with endovascular coiling are examined regularly with angiography, and if the coil mass has compacted or the aneurysm shows signs of re-growth, additional coils are inserted into the aneurysm to stabilize the aneurysm occlusion. Follow-up examinations of aneurysms treated with coils suggest that an increased packing density of coils results in reduced compaction events and better treatment outcomes. The packing density of the coil, which is the ratio of the volume of coils inserted into the aneurysm sac and the volume of the aneurysm sac, may therefore be used as a measure of efficacy of the treatment. Maximum coil packing densities that may be achieved with the two-dimensional structures were approximately 25-30%, but those values have increased to approximately 35-40% with the more convoluted three-dimensional structures.

Endovascular stents have been developed to buttress coil masses within the aneurysm sacs and slightly higher packing densities (approximately 45%) may be achieved with stent-assistance. In general, much lower packing densities have been achievable in larger aneurysms of 10-20 mm diameter. One explanation for these low packing densities (less than half of the aneurysm volume is being filled) is that the orientation of these three-dimensional coil structures is more or less random within the aneurysm. This results in overlapping of the coil wires such that the interstices between and around such overlaps cannot be accessed and filled with subsequently inserted coils.

Therefore, what is needed is a device and method for packing and effectively occluding aneurysm sacs to maximize the packing density and occlude the aneurysm.

### SUMMARY OF THE INVENTION

The present invention advantageously provides a method and device for occluding an aneurysmal sac with substantially spherical helix coils. The device may comprise a wire defining a first substantially spherical helix, the wire being releaseably engageable with a medical device.

In another embodiment of the present invention, the device may comprise a first wire having a shape memory, the first wire defining a first substantially spherical helix. A second wire may be included having a shape memory, the second wire defining a second substantially spherical helix nested within the first substantially spherical helix. A catheter may be releaseably engageable with the first and second wires, the wires being at least partially disposable within the catheter.

In yet another embodiment of the present invention, the method includes positioning a wire proximate to an opening defined by the aneurysmal sac. The wire may then be deployed within the aneurysmal sac, such that the wire defines a first substantially spherical helix within the aneurysmal sac.

In yet another embodiment of the present invention, the method includes providing a first wire having a shape memory and a diameter in the range of 50 to 100 microns (μm). The first wire may further define a coil having a diameter in the range of 100 to 500 μm. The first wire may then be releaseably engaged to a catheter. The first wire may then be positioned proximate an opening defined by the aneurysmal sac. The first coil may then be deployed within the aneurysmal sac, such that as the wire is released from the distal end of the catheter it defines a first substantially spherical helix inside the aneurysm. A second coil may be provided having a shape memory and a diameter in the range of approximately 50 to 100 μm, the second wire defining a coil having a diameter in the range of 100 to 500 μm. The second coil may be releaseably engaged to the catheter. The second wire may then be positioned proximate the opening. The second coil may then be deployed within the aneurysmal sac, such that the second coil defines a second substantially spherical helix nested within the first substantially spherical helix.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a perspective view of an embodiment of the wire of the present invention;

FIG. 2 shows a perspective view of the centerline an embodiment of a first substantially spherical helix of the present invention;

FIG. 3 shows an embodiment of the substantially spherical helix of FIG. 2 with a second substantially spherical helix nested within the first substantially spherical helix;

FIG. 4 shows the centerline of the first substantially spherical helix deployed within an aneurysm;

FIG. 5a shows a front view of the substantially spherical helix of FIG. 2 being deployed within the aneurysmal sac with a catheter;

FIG. 5b shows a front view of the substantially spherical helix of FIG. 2 being buttressed by a stent in the vasculature.

FIG. 6 shows a front view of a stent buttressing a plurality of spherical helices of FIGS. 5a and 5b.

### DETAILED DESCRIPTION OF THE INVENTION

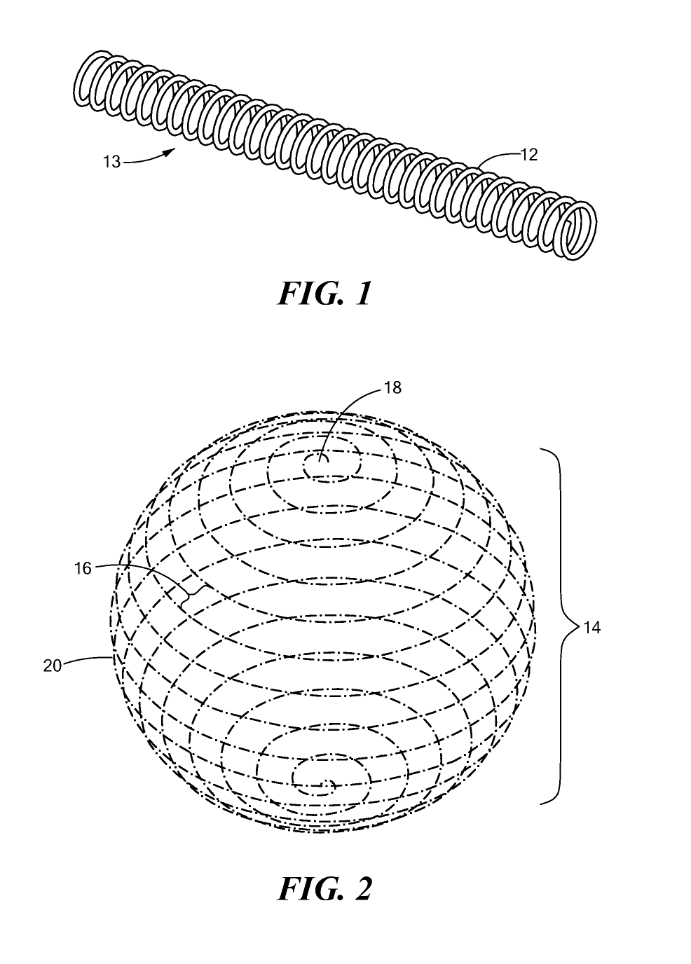

Referring now to the drawings in which like reference designators refer to like elements, there is shown in FIG. 1 an exemplary embodiment of an aneurysmal occlusion device in accordance with the principles of the present invention. The device may include a wire 12 having a shape memory, such that the wire 12 may be sufficiently malleable to be manipulated to a first shape, and retain the first shape until manipulated to a second shape. For example, the wire 12 may be comprised of a shape memory and embolic biocompatible material such as Nitinol. Alternatively, the wire 12 may be comprised of thermoplastic material, polymers, or material responsive to an electromagnetic current, such that when deployed, the wire 12 may deform, define, or otherwise be configured to a desired structure.

The cross-section of the wire 12 may be circular, or any other shape, such as rectangular, oval, or triangular. The cross-sectional diameter of the wire 12 may be, for example, approximately 50 to 100 μm or any diameter. In one embodiment, the wire 12 may define, or be fabricated to define a coil 13. The coil 13 may be substantially helical and may be fabricated or defined by winding, bending, or otherwise deforming the wire 12 about a mandrel or other object. The mandrel may be removed after coiling the wire 12 resulting in the coil 13 having a diameter in the range of about 100-500 μm.

Referring now to FIG. 2, the coil 13 or wire 12 may then be fabricated to define a substantially spherical helix. For example, the coil 13 may be wound about a spherical mold having particular diameter. The coil 13 may then be heated or cooled such that it plastically or otherwise deforms about the spherical mold to define a first substantially spherical helix 14. The first substantially spherical helix 14 may then be removed from the mold such that it retains its shape.

Continuing to refer to FIG. 2, the first substantially spherical helix 14 may further define a plurality of cross-sectional diameters. For example, the first substantially spherical helix 14 may define two-dimensional planes 18 and an equator 20, wherein the poles 18 have the smallest diameters, which may be at least approximately 0.5 mm, and the equator 20 has the largest diameter, which may be at most approximately 40 mm depending on the dimension of the aneurysm. The centerline of wire 12 or coil 13 is shown to be tightly wound, wherein each 360 degree rotation of the coil 13 is separated longitudinally 16 by the diameter of one coil 13, approximately 300 microns.

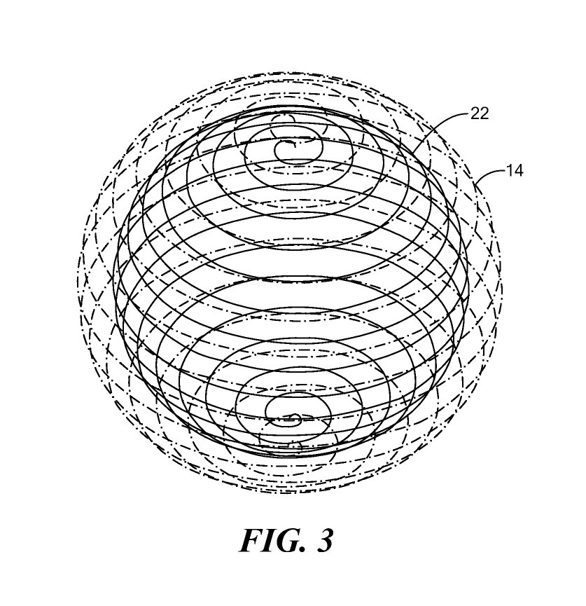

Referring now to FIG. 3, if additional spherical helices are needed to fill a volume defined by an aneurism, the wire 12, coil 13, or additional wires, may be deployed about or within the first substantially spherical helix 14 to form a second substantially spherical helix 22 and/or a plurality of additional spherical helices or other non-helical substantially spherical structures. In an embodiment, approximately 10-15 spherical helices are deployed and defined within an aneurismal volume to fill and occlude the aneurism. The second substantially spherical helix 22 or any additionally spherical helices may be concentric to and nested within the first substantially spherical helix 14, which may further minimize any interstitial space between the first and second spherical helices. The second substantially spherical helix 22 may be fabricated in a similar manner to that of the first substantially spherical helix 14. For example, if the second substantially spherical helix 22 has a smaller diameter than the first substantially spherical helix 14, then the second substantially spherical helix 22 may be fabricated by winding and deforming the wired coil 13 about a spherical mold having a smaller diameter than the spherical mold used to fabricated the first substantially spherical helix 14.

As the spherical helices are successively nested within the aneurysm, each spherical helix may apply an outward force against other nested spherical helices. This outward force may cause the nested spherical helices to become tightly packed and reduce the formation of interstitial space. Moreover, after the nested spherical helices are deployed, the natural biological response to the nested spherical helices may be to release clotting agents or other biological products. These biological products may act like a glue and form scar tissue around the nested spherical helices, which may help to maintain the spheres' stability and reduce the formation of interstitial space for an extended period of time.

Alternatively, the second substantially spherical helix 22 may be positioned adjacent or proximate to the first substantially spherical helix 14 to accommodate different shapes and sizes of aneurysms. The volumes of the first and second spherical helices may be substantially equal or different. For example, the second substantially spherical helix 22 may be wound concentrically about the first substantially spherical helix 14, resulting in the second substantially spherical helix 22 defining a larger volume than the first spherical helix 14. Alternatively, the second substantially spherical helix 22 may be wound within the first substantially spherical helix 14, resulting in the first substantially spherical helix 14 defining a larger volume than the second substantially spherical helix 22.

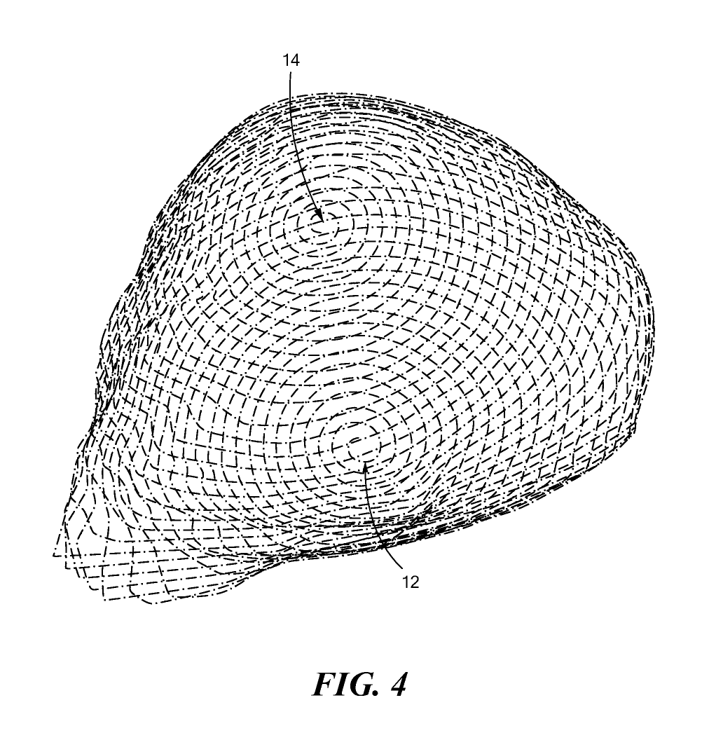

Referring now to FIG. 4, where a model of a simulated aneurysm is shown. The aneurysm diameter (average width and height) is approximately 10 mm. The volume of the aneurysm is approximately 535 mm³. The coil 13 may be wound to fill a substantial portion of the volume defined by the aneurysm. The centerline of wire 12 or coil 13 is shown to be tightly wound, wherein each 360 degree rotation of the coil 13 is separated longitudinally by the diameter of one coil 13, approximately 300 microns. As shown, the first substantially spherical helix 14 occupies approximately 13% of the aneurysmal volume. Filling the remainder of the aneurysmal volume with concentric spherical helices may result in a final packing density of within the aneurysm of approximately 72%. Alternatively, if the aneurysm were a sphere, the final packing density may be approximately 78% or higher, irrespective of the diameter of the aneurysm.

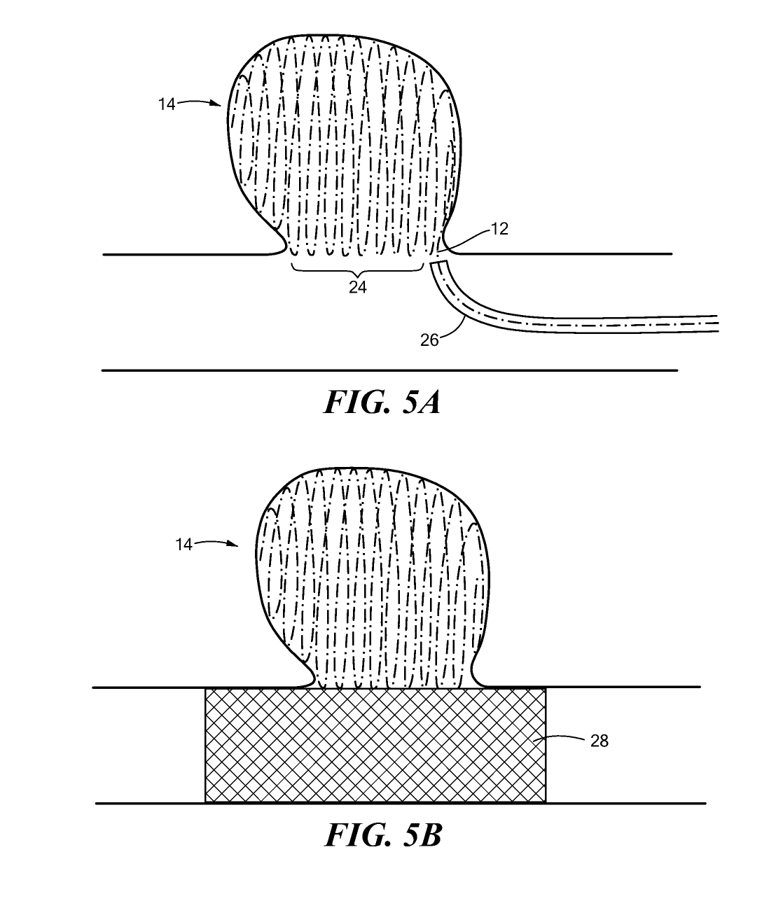

Now referring to FIG. 5A, the coil 13 may be positioned proximate to an opening 24, known as the neck, by the use of a medical device 26. The medical device 26 may be for example, a catheter or any other endovascular device capable of deploying coil 13 within the aneurysmal volume. The medical device 26 may define a proximal end, a distal end, and a catheter lumen for insertion of a guide wire, pusher wire, or the coil 13. The wire 12 or coil 13 may be releaseably engageable to a portion of the guide wire or may be threaded independently through the catheter lumen. For example, the coil 13 may define a loop or other attachment element that is engageable with a distal portion of the guide wire. The attachment element may be actuated, by methods known in the art, to detach the coil 13 from the guide wire. The coil 13 may further be biased when disposed within the medical device 26. For example, the coil 13 may be substantially longitudinal when biased within the catheter lumen. As the coil 13 is deployed within the aneurismal sac, the coil 13 may unwind, deform, or otherwise be configured to define the first substantially spherical helix 14.

Now referring to FIG. 5B, a stent 28 may be deployed in the parent vessel straddling the aneurysm neck. For example, the stent 28 may be releaseably engaged to the distal end of a catheter. The stent 28 may be any expandable stent used to buttress coils deployed in cerebral aneurysms. Before, during, or after deployment of the first substantially spherical helix 14, the stent 28 may be deployed proximate the opening 24 to buttress the first substantially spherical helix 14 and may prevent the first substantially spherical helix 14 from migrating out of the aneurysm or otherwise deforming into the vasculature.

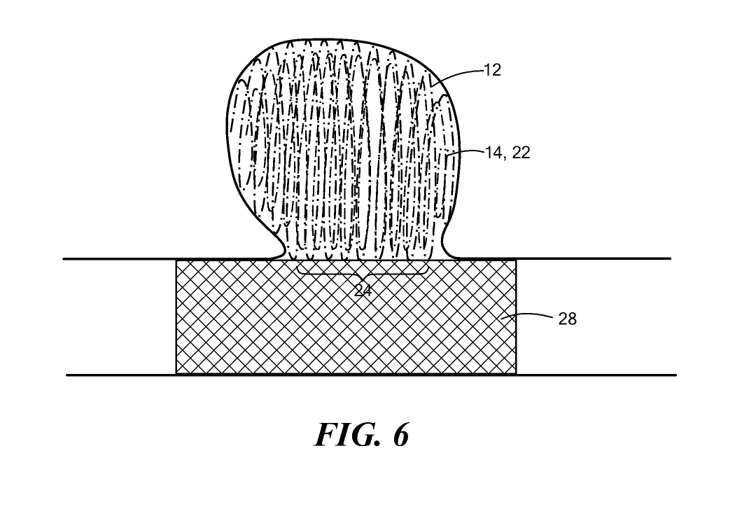

Referring now to FIG. 6, if additional spherical helices are needed to fill a volume defined by an aneurysm, additional coils may be fabricated as discussed above to form a spherical helix, but with a different dimension, may be deployed within the first substantially spherical helix 14 to form a second substantially spherical helix 22, resulting in the first substantially spherical helix 14 defining a larger volume than the second substantially spherical helix 22. Additional wires may be deployed within the first and second substantially spherical helices to form a plurality of additional spherical helices or other non-helical substantially spherical structures. In an embodiment, approximately 10-15 spherical helices are deployed within an aneurysmal volume to fill and occlude the aneurysm. The second substantially spherical helix 22 or any additionally spherical helices may be concentric to and nested within the first substantially spherical helix 14, which may further minimize any interstitial space between all the spherical helices.

As the spherical helices are successively nested within the aneurysm, each spherical helix may apply an outward force against other nested spherical helices. This outward force may cause the nested spherical helices to become tightly packed and reduce the formation of interstitial space. Moreover, after the nested spherical helices are deployed, the natural biological response to the nested spherical helices may be to form thrombi from activated clotting agents residing in low blood flow zones. These thrombi may eventually progress to form scar tissue around and within the nested spherical helices, which may help to maintain the spheres' stability and reduce the formation of interstitial space for an extended period of time.

In addition to a medical device, the present invention provides for a method for occluding the opening 24 of the aneurysm. The method may include providing the wire 12 of diameter approximately 50 to 100 μm, or any diameter, wound helically to define a coil 13. The diameter of the coil 13 may be for example, approximately 100 to 500 μm, or any diameter. The method may further include releaseably engaging the coil 13 to the medical device 26, for example a catheter. The coil 13 may be releaseably engaged to the catheter before, during, or after insertion of the medical device 26 within the vasculature. The coil 13 may further define a first substantially spherical helix 14, such that the first substantially spherical helix 14 may be stretched or biased when engaged within the medical device 26.

The coil 13 may then be positioned proximate to the opening 24 defined by the aneurysmal sac. The coil 13 may then be deployed within the aneurysmal sac by releaseably engaging the coil 13 to a guide wire and feeding the guide wire and coil 13 into the aneurysm sac. During deployment of the coil 13 within the aneurysm, the coil 13 may unwind or otherwise return to its pre-shaped configuration of the substantially spherical helix 14.

After deploying the first substantially spherical helix 14 within the aneurysm, the coil 13 may be disengaged from the medical device 26. Additional coil or wires may then be releaseably engaged to the medical device 26 for deployment in the aneurysm. The coil 13 or additional coils may then be deployed within the aneurysm and define a second or a plurality of substantially spherical helices 22 concentric to the first substantially spherical helix 14. Additional substantially spherical helices may be defined concentric to or adjacent to the first substantially spherical helix to fill and occlude the aneurysm. After the spherical helices have been defined by coil 13 or additional wires, the coil 13 or additional wires may be disengaged from the medical device 26. Concomitantly or consecutively to the coil 13 or additional wires being disengaged from the medical device 26, a stent 28 may be positioned proximate the opening 24 to buttress the spherical helices within the aneurysmal sac. The buttressing of the stent 28 against the opening 24 may prevent the spherical helices from herniating into the vasculature.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. An aneurysmal occlusion system comprising:
a first wire having a shape memory defining a coil, the first wire defining a first substantially spherical helix;
a second wire having a shape memory defining a coil, the second wire defining a second substantially spherical helix nested within the first substantially spherical helix; and
a catheter releaseably engageable with the first and second wires, the first and second wires being at least partially disposable within the catheter.

2. (canceled)

3. The aneurysmal occlusion system of claim 1, wherein the first wire and second wire each has a diameter of approximately 50 to 100 μm.

4. (canceled)

5. (canceled)

6. The aneurysmal occlusion device of claim 3, wherein the coil has a diameter of approximately 100 to 500 μm.

7. (canceled)

8. The aneurysmal occlusion device of claim 1, wherein at least one cross-section of the first substantially spherical helix has a diameter of approximately 0.5 mm.

9. The aneurysmal occlusion device of claim 1, wherein at least one cross-section of the first substantially spherical helix has a diameter of approximately 40 mm.

10. (canceled)

11. A method of occluding an aneurysmal sac comprising:
positioning a first wire proximate an opening defined by the aneurysmal sac;
deploying the first wire within the aneurysmal sac, such that the wire defines a first substantially spherical helix within the aneurysmal sac;
positioning a second wire proximate the opening;
deploying the second wire within first wire within the aneurysmal sac, such that the second wire defines a second substantially spherical helix nested within the first substantially spherical helix.

12. The method of claim 11, further including releaseably engaging the wire to a catheter.

13. (canceled)

14. The method of claim 11, wherein the wire has a shape memory.

15. The method of claim 14, wherein the wire defines a coil.

16. The method of claim 11, wherein at least one cross-section of the first substantially spherical helix has a diameter of approximately 0.5 mm.

17. The method of claim 11, wherein at least one cross-section of first substantially spherical helix has a diameter of approximately 40 mm.

18. The method of claim 11, wherein the wire has a diameter of approximately 50 to 100 μm.

19. The method of claim 15, wherein the coil has a diameter of approximately 100 to 500 μm.

20. A method of occluding an aneurysmal sac comprising:
providing a first wire having a shape memory and a diameter of approximately 50 to 100 μm, the first wire defining a coil having a diameter of 100 to 500 μm;
releaseably engaging the first wire to a catheter;
positioning the first wire proximate an opening defined by the aneurysmal sac;
deploying the first wire within the aneurysmal sac, such that the first wire defines a first substantially spherical helix;
providing a second wire having a shape memory and a diameter of approximately 50 to 100 μm, the second wire defining a coil having a diameter of 100 to 500 μm;
releaseably engaging the second wire to the catheter;
positioning the second wire proximate an opening defined by the aneurysmal sac; and
deploying the second wire within the aneurysmal sac, such that the second wire defines a second substantially spherical helix nested within the first substantially spherical helix.
